(19) United States
(12) Patent Application Publication
(10) Pub. No.: US 2026/0363809 A1
(43) Pub. Date: Nov. 19, 2026

(54) SYSTEMS AND METHODS FOR CREATING VIRTUAL BOUNDARIES IN ROBOTIC SURGICAL SYSTEMS

(71) Applicant: COVIDIEN LP, Mansfield, MA (US)

(72) Inventors: Michael A. Eiden, Somerville, MA (US); Tuvia C. Rappaport, Somerville, MA (US); Max L. Balter, Boston, MA (US); Zachary A. Walker-Liang, Kensington, MD (US)

(21) Appl. No.: 19/136,370

(22) PCT Filed: Dec. 13, 2023
(86) PCT No.: PCT/IB2023/062627

§ 371 Date: Jun. 6, 2025

Related U.S. Application Data
(60) Provisional application No. 63/432,432, filed on Dec. 14, 2022.

(51) Int. Cl. A61B 34/00; A61B 34/20; A61B 34/37; A61B 90/00; G16H 40/67

(52) U.S. Cl. CPC A61B 34/76; A61B 34/37; G16H 40/67; A61B 2034/2065; A61B 2090/365

(57) ABSTRACT

A system for clinical workspace augmentation includes an augmented reality device. The augmented reality headset includes an imaging device configured to capture images of a real-world environment, a display configured to display a composite view, a processor, and a memory. The memory includes instructions stored thereon which, when executed by the processor, cause the system to identify an input object in the captured real-world environment; track a path of the input object in the captured real-world environment; generate a boundary zone based on the tracked path of the input object in the captured real-world environment; render an overlay including the boundary zone; and display a composite view of the captured real-world environment and the overlay on a display of an augmented reality headset.

This application claims the benefit of U.S. Provisional Patent Application Ser. No. 63/432,432, filed Dec. 14, 2022, the entire content of which is incorporated herein by reference.

### BACKGROUND

### Technical Field

The disclosure generally relates to systems and methods for workspace augmentation. In particular, the present disclosure is directed to a virtual or augmented reality simulated setup of robotic surgical systems with boundary zones.

### Background of Related Art

Robotic surgical systems are used in minimally invasive medical procedures. Some robotic surgical systems include a surgical console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a worksite within the patient's body.

However, in some conventional systems, while a robotic arm is moving around, the robotic arm may move “outside” physical or virtual environments, which degrades the user experience.

### SUMMARY

In accordance with aspects of the disclosure, a computer-implemented method for clinical workspace augmentation is presented. The method includes capturing a real-world environment including an input object by an imaging device; identifying the input object in the captured real-world environment; tracking a path of the input object in the captured real-world environment; generating at least one boundary zone based on the tracked path of the input object in the captured real-world environment; rendering an overlay including the at least one boundary zone; and displaying a composite view of the captured real-world environment and the overlay on a first display of an augmented reality device.

In an aspect of the disclosure, the input object may include a hand of a user and/or an input controller.

In another aspect of the disclosure, tracking the path of the input object may be based on a gesture, pose recognition, eye gaze tracking, and/or voice command.

In yet another aspect of the disclosure, identifying the input object in the captured real-world environment may be based on object detection.

In a further aspect of the disclosure, object detection may be performed by: generating a spatial mesh based on the captured real-world environment; determining boundaries of the input object based on the spatial mesh; and identifying the input object based on a machine learning model, where the determined boundaries are provided as an input to the machine learning model.

In yet a further aspect of the disclosure, the method may further include modifying the at least one boundary zone by: selecting a generated shape configured to become a portion of the at least one boundary zone; positioning the generated shape in the captured real-world environment; receiving input to transform the generated shape by moving, rotating, and/or scaling the generated shape; and adding the transformed generated shape to the at least one boundary zone.

In yet a further aspect of the disclosure, the method may further include modifying the at least one boundary zone by: selecting a plurality of waypoints to become a portion of the at least one boundary zone; receiving input to position the plurality of waypoints; connecting the waypoints from a second boundary zone; and adding the second boundary zone to the at least one boundary zone.

In another aspect of the disclosure, the method may further include receiving 3D volumetric data of a robotic arm of a robotic surgical system in the real-world environment; receiving a spatial position of the robotic arm; determining that the robotic arm is crossing a threshold of the at least one boundary zone based on the spatial position and the 3D volumetric data; and stopping the robotic arm from entering the at least one boundary zone.

In yet a further aspect of the disclosure, the method may further include providing force feedback to an input device of a surgical console of the robotic surgical system in response to the determining that the robotic arm is crossing the threshold of the at least one boundary zone.

In another aspect of the disclosure, the method may further include displaying on a second display a composite view of the at least one boundary zone and the real-world environment from a point of view of the second display. The point of view of the second display is different than a point of view of the first display.

In accordance with aspects of the disclosure, a system for clinical workspace augmentation that includes an augmented reality device is presented. The augmented reality device includes an imaging device configured to capture images of a real-world environment, a display configured to display a composite view, a processor, and a memory. The memory includes instructions stored thereon which, when executed by the processor, cause the system to identify an input object in the captured real-world environment; track a path of the input object in the captured real-world environment; generate at least one boundary zone based on the tracked path of the input object in the captured real-world environment; render an overlay including the at least one boundary zone; and display a composite view of the captured real-world environment and the overlay on a first display of an augmented reality device.

In an aspect of the disclosure, the input object may include a hand of a user and/or an input controller.

In another aspect of the disclosure, tracking the path of the input object may be based on a gesture, pose recognition, eye gaze tracking, and/or voice command.

In yet another aspect of the disclosure, identifying the input object in the captured real-world environment may be based on object detection.

In a further aspect of the disclosure, the object detection may be performed by generating a spatial mesh based on the captured real-world environment; determining boundaries of the input object based on the spatial mesh; and identifying the input object based on a machine learning model, where the determined boundaries are provided as an input to the machine learning model.

In a further aspect of the disclosure, the instructions, when executed by the processor, may further cause the system to modify the at least one boundary zone by: selecting a generated shape configured to become a portion of the at least one boundary zone; positioning the generated shape in the captured real-world environment; receiving input to transform the generated shape by moving, rotating, and/or scaling the generated shape; and adding the transformed generated shape to the at least one boundary zone.

In yet a further aspect of the disclosure, the instructions, when executed by the processor, may further cause the system to modify the at least one boundary zone by selecting a plurality of waypoints to become a portion of the at least one boundary zone; receiving input to position the plurality of waypoints; connecting the waypoints from a second boundary zone; and adding the second boundary zone to the at least one boundary zone.

In an aspect of the disclosure, the instructions, when executed by the processor, may further cause the system to: receive 3D volumetric data of a robotic arm of a robotic surgical system in the real-world environment; receive a spatial position of the robotic arm; determine that the robotic arm is crossing a threshold of the at least one boundary zone based on the spatial position and the 3D volumetric data; and stop the robotic arm from entering the at least one boundary zone.

In another aspect of the disclosure, the instructions, when executed by the processor, may further cause the system to provide force feedback to an input device of a surgical console of the robotic surgical system in response to the determining that the robotic arm is crossing the threshold of the at least one boundary zone.

In accordance with aspects of the disclosure, a non-transitory computer-readable medium is presented. The non-transitory computer-readable medium stores instructions which, when executed by a processor, cause the processor to perform a method that includes capturing by an imaging device a real-world environment that includes an input object; identifying the input object in the captured real-world environment; tracking a path of the input object in the captured real-world environment; generating a at least one boundary zone based on the tracked path of the input object in the captured real-world environment; rendering an overlay including the at least one boundary zone; and displaying a composite view of the captured real-world environment and the overlay on a first display of an augmented reality device.

### BRIEF DESCRIPTION OF THE DRAWINGS

Various aspects of the disclosure are described herein with reference to the drawings wherein:

FIG. 1 is a schematic illustration of a robotic surgical system including a control tower, a console, and one or more surgical robotic arms according to an aspect of the disclosure;

FIG. 2 is a perspective view of a surgical robotic arm of the robotic surgical system of FIG. 1 according to an aspect of the disclosure;

FIG. 3 is a perspective view of a setup arm with the surgical robotic arm of the robotic surgical system of FIG. 1 according to an aspect of the disclosure;

FIG. 4 is a schematic diagram of a computer architecture of the robotic surgical system of FIG. 1 according to an aspect of the disclosure;

FIG. 5 is a schematic view of the robotic surgical system of FIG. 1 positioned about a surgical table according to an embodiment of the present disclosure;

FIG. 6 is a flow chart for a computer-implemented method for clinical workspace augmentation according to an aspect of the disclosure; and

FIG. 7 is an image of a composite view of the clinical workspace augmentation displaying a boundary according to an aspect of the disclosure.

### DETAILED DESCRIPTION

Aspects of the presently disclosed robotic surgical system are described in detail with reference to the drawings, in which like reference numerals designate identical or corresponding elements in each of the several views. As used herein, the term “distal” refers to the portion of the robotic surgical system and/or the surgical instrument coupled thereto that is closer to the patient, while the term “proximal” refers to the portion that is farther from the patient.

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on a controller or on a user device, including, for example, a mobile device, a personal computer, or a server system.

As will be described in detail below, the disclosure is directed to a robotic surgical system including a surgical console, a control tower, and one or more movable carts having a surgical robotic arm coupled to a setup arm. The surgical console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command.

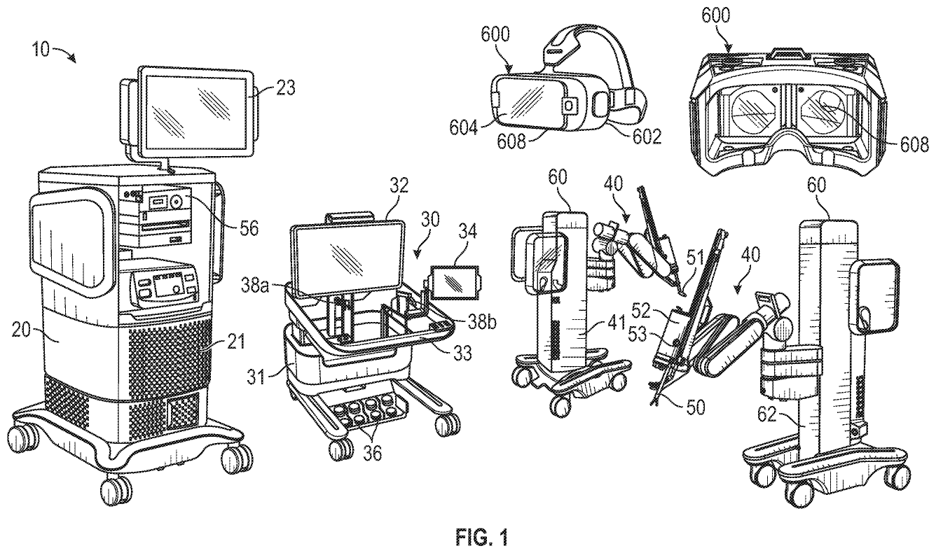

With reference to FIG. 1, a robotic surgical system 10 generally includes an augmented reality device 600 (e.g., AR headset), a control tower 20, which is connected to all of the components of the robotic surgical system 10, including a surgical console 30 and one or more robotic arms 40. Each of the robotic arms 40 includes a surgical instrument 50 removably coupled thereto. Each of the robotic arms 40 is also coupled to a movable cart 60.

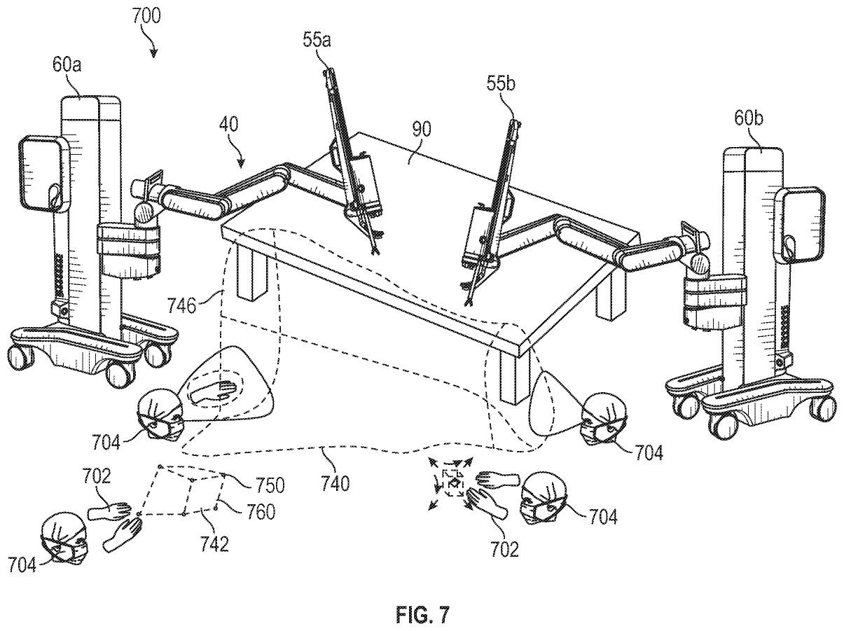

The augmented reality device 600 is configured to display a composite view and generally includes a controller 602, an imaging device 604, and a display 608. The controller 602 includes a memory configured to have instructions stored thereon and a processor configured to execute the instructions. The augmented reality device 600 may overlay virtual objects such as a virtual robot arm (FIG. 7). For example, the augmented reality device 600 can provide users advice on how to position various virtual objects to help set up an operating room for a surgery. It is contemplated that the augmented reality device 600 may be full virtual reality such as the Quest Pro® from Meta®, of Menlo Park, CA or an augmented reality (mixed reality) headset such as HoloLens® from Microsoft®, of Seattle, WA.

The surgical instrument 50 is configured for use during minimally invasive surgical procedures. In aspects, the surgical instrument 50 may be configured for open surgical procedures. In aspects, the surgical instrument 50 may be an endoscope, such as an endoscopic camera 51, configured to provide a video feed for the user. In further aspects, the surgical instrument 50 may be an electrosurgical forceps configured to seal tissue by compressing tissue between jaw members and applying electrosurgical current thereto. In yet further aspects, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include the endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 perform the image processing based on the depth estimating algorithms of the disclosure and output the processed video stream.

The surgical console 30 includes a first display 32, which displays a video feed of the surgical site provided by camera 51 of the surgical instrument 50 disposed on the robotic arms 40, and a second display 34, which displays a user interface for controlling the robotic surgical system 10. The first and second displays 32 and 34 are touchscreens allowing for displaying various graphical user inputs.

The surgical console 30 also includes a plurality of user interface devices, such as foot pedals 36 and a pair of handle controllers 38a and 38b which are used by a user to remotely control robotic arms 40. The surgical console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

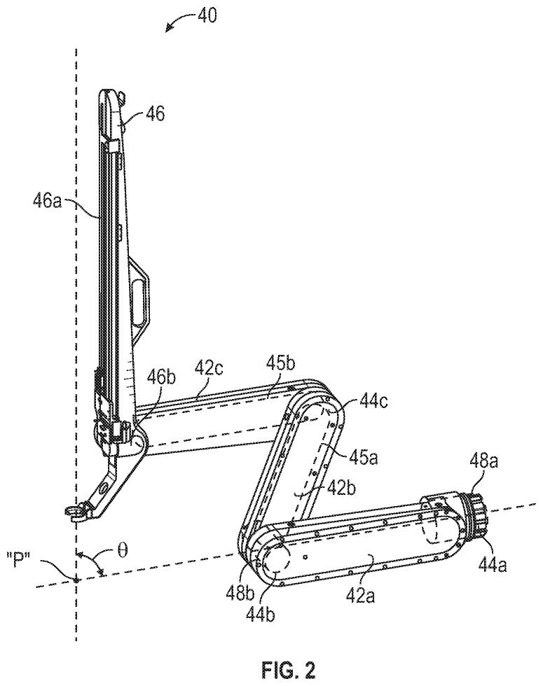

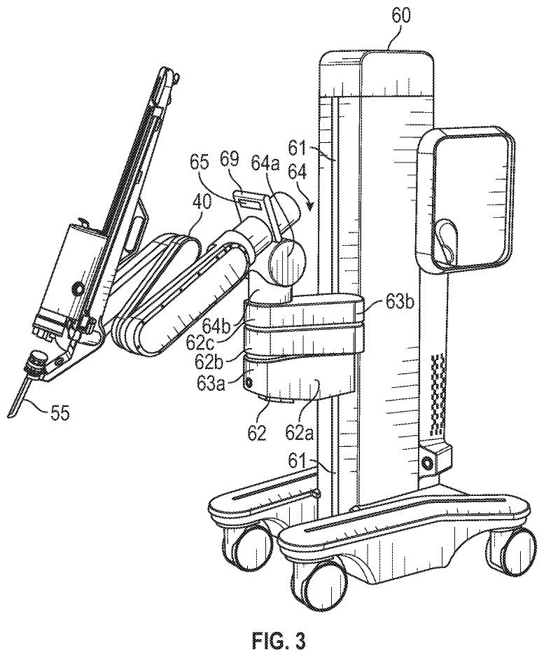

With reference to FIG. 2, each of the robotic arms 40 may include a plurality of links 42a, 42b, 42c, which are interconnected at joints 44a, 44b, 44c, respectively. The joint 44a is configured to secure the robotic arm 40 to the movable cart 60 and defines a first longitudinal axis. With reference to FIG. 3, the movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40.

The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In aspects, the robotic arm 40 may be coupled to the surgical table (not shown). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b, such that the actuator 48b is configured to rotate each of the links 42b, 42c and the holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In aspects, some, or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the robotic arm 40 also includes a holder 46 defining a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic port 55 (FIG. 3) held by the holder 46.

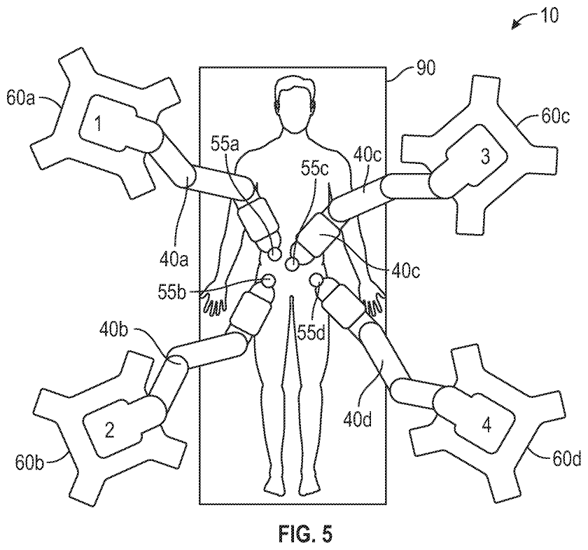

The robotic arm 40 also includes a plurality of manual override buttons 53 (FIGS. 1 and 5) disposed on the IDU 52 and the setup arm 62, which may be used in a manual mode. The user may press one or more of the buttons 53 to move the component associated with the button 53.

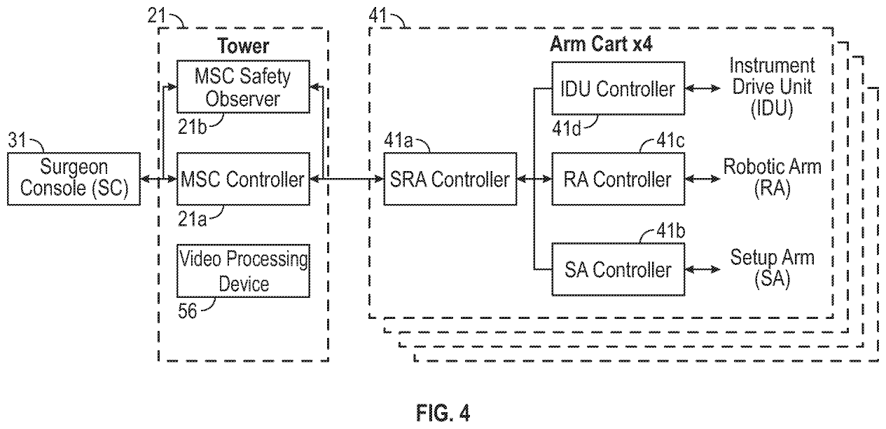

With reference to FIG. 4, each of the computers 21, 31, 41 of the robotic surgical system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgical console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the robotic surgical system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

The setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand-eye transform function executed by the controller 21a. The hand-eye function, as well as other functions described herein, is/are embodied in software executable by the controller 21a or any other suitable controller described herein. The pose of one of the handle controller 38a may be embodied as a coordinate position and role-pitch-yaw (“RPY”) orientation relative to a coordinate reference frame, which is fixed to the surgical console 30. The desired pose of the instrument 50 is relative to a fixed frame on the robotic arm 40. The pose of the handle controller 38a is then scaled by a scaling function executed by the controller 21a. In aspects, the coordinate position is scaled down and the orientation is scaled up by the scaling function. In addition, the controller 21a also executes a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

The video processing device 56 is configured to process the video feed from the endoscope camera 51 and to output a processed video stream on the first displays 32 of the surgical console 30 and/or the display 23 of the control tower 20.

With reference to FIG. 5, the robotic surgical system 10 is setup around a surgical table 90. The system 10 includes mobile carts 60a-d, which may be numbered “1” through “4.” During setup, each of the carts 60a-d are positioned around the surgical table 90. Position and orientation of the carts 60a-d depends on a plurality of factors, such as placement of a plurality of access ports 55a-d, which in turn, depends on the surgery being performed. Once the port placement is determined, the access ports 55a-d are inserted into the patient, and carts 60a-d are positioned to insert instruments 50 and the laparoscopic camera 51 into corresponding ports 55a-d.

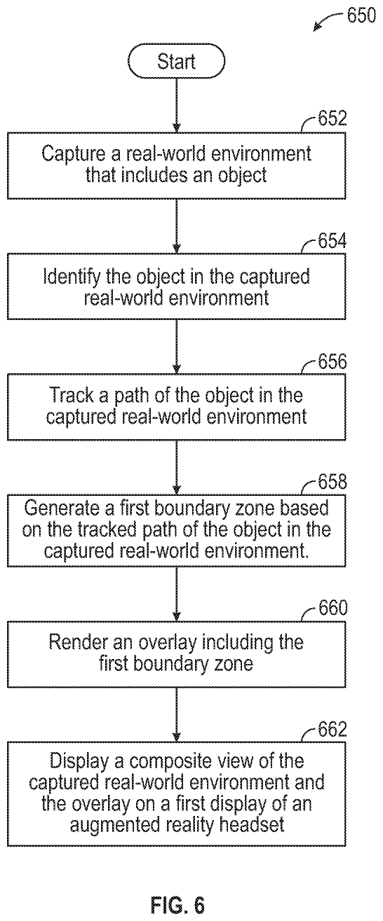

FIG. 6 shows a flow chart illustrating various operations of an exemplary method for clinical workspace augmentation with boundary zones. Persons skilled in the art will appreciate that one or more operations of the method 650 may be performed in any suitable order, repeated, and/or omitted without departing from the scope of the disclosure. In various aspects, the illustrated method 650 can operate in one or more controllers, i.e., controllers 21a, 31a, 41a, 602, in a remote device, or in another server or system. The operations may be embodied as software or instructions executable by the controller. Although the operations of method 650 are described below with respect to the controller 602 (FIG. 1) of augmented reality device 600 (FIG. 1), but other components and controllers of the system 10 may be used. The method 650 is described with respect to FIG. 7 which shows a composite view of the clinical workspace.

Initially, at step 652, the controller 602 causes the robotic surgical system 10 to capture a real-world environment by an imaging device 604 of an augmented reality device 600 (FIG. 1) or any other imaging device (e.g., coupled to a mobile device/tablet). The imaging device 604 may include a stereoscopic imaging device. The real-world environment includes an input object 702. The input object 702 may be a hand of a user (e.g., a clinician) and/or an input controller. The input object 702 is used to draw a virtual boundary as described in further detail below. An input controller may include a set of sensors for tracking the position of the controllers. For example, the tracking may include optical tracking using the imaging device 604 of augmented reality device 600. The input controller may include a set of infrared LEDs located, for example, on the rings of the input controller. The imaging device 604 detects the LEDs and then the controller 602 triangulates the position of the controllers in space.

Next, at step 654, the controller 602 causes the robotic surgical system 10 to identify the input object 702 in the captured real-world environment. For example, the input object 702 may be identified as a hand. Real-world objects, such as the patient, the user, the input object 702, and/or the surgical table 90, may be detected using edge detection and/or image segmentation. For example, the controller 602 may extract the edges of the input object 702 in the captured image, by detecting discontinuities in depth, discontinuities in surface orientation, and/or changes in material properties of the input object 702 in the captured image. The extracted edges may be used to determine the boundaries of the input object 702. The controller may then identify the input object 702 based on the determined boundaries, for example, as the hand.

In aspects, identifying the input object 702 in the captured real-world environment may be based on object detection. The object detection may be performed by generating a spatial mesh based on the captured real-world environment, determining boundaries of the object based on the spatial mesh, and identifying the input object 702 based on a machine learning model. The determined boundaries may be provided as an input to the machine learning model. The machine learning model may be trained on labeled images of objects, e.g., hands.

Next, at step 656, the controller 602 enters a boundary generation mode during which a user creates a virtual boundary (e.g., a first boundary zone 740 of FIG. 7) that is used to track movement of the robotic arms 40 and other objects. The boundary may be created by the robotic surgical system 10 to track a path 746 of the input object 702 in the captured real-world environment. For example, a user can “paint” custom trails, virtually, by moving their hand, a 3D trail follows the movement of the input object 702. Tracking the path 746 of the object may be based on at least one of a gesture recognition, pose recognition, eye gaze tracking, and/or voice command, and may be done in 3D. The resulting path 746 may be adjustable using the input object 702. The path 746 may have one or more waypoints that are movable by the input object 702 to modify the length, direction, shape, curvature, etc. of the path.

The controller 602 may cause the robotic surgical system 10 to detect the width and/or volume of the input object 702. For example, the tracked path 746 could include a volume and/or a width of the user's hand.

Next, at step 658, the controller 602 causes the robotic surgical system 10 to generate a first boundary zone 740 (FIG. 7) based on the tracked path 746 of the input object 702 in the captured real-world environment. The boundary zone 740 may automatically connect to itself in the case where it is not drawn as a completed and/or closed shape.

In aspects, the user 704 can place generated shapes 742 (e.g., cubes, spheres, etc.) and move, scale, and/or rotate them into place to form custom boundary zones 740 and/or modify existing boundary zones 740. The controller 602 may cause the robotic surgical system 10 to modify the first boundary zone 740 by selecting a generated shape 742 configured to become a portion of the first boundary zone; positioning the generated shape 742 in the captured real-world environment; receiving input to transform the generated shape 742 by moving, rotating, and/or scaling the generated shape 742; and adding the transformed generated shape 742 to the boundary zone. In aspects, multiple boundary zones may be generated.

The user can place waypoints 750 that will connect to form a custom boundary zone or to modify an existing boundary zone. The controller 602 may cause the robotic surgical system 10 to modify the first boundary zone 740 by selecting a plurality of waypoints 750 to become a portion of the first boundary zone 740. The controller 602 may receive input to position the plurality of waypoints. The controller 602 may connect the waypoints from a second boundary zone 760 and add the second boundary zone 760 to the first boundary zone 740.

In aspects, object detection may be used to identify a person (e.g., a clinician), and the controller 602 may cause the robotic surgical system 10 to generate a moving boundary around the person (e.g., prevent collisions with a bedside assistant).

Next, at step 660, the controller 602 causes the robotic surgical system 10 to render an overlay that includes the first boundary zone 740. At step 662, the controller 602 causes the robotic surgical system 10 to display a composite view 700 (FIG. 7) of the captured real-world environment and the overlay on a display 608 of an augmented reality device 600 (FIG. 1).

One or more users can view the boundary zone 740 via the augmented reality device 600 or another display and/or projection device. For example, a user, such as the bedside assistant, could see the area, as displayed on the display 608 of an augmented reality device 600, which is reserved for the anesthesiologist (or any other clinical staff) and would know to avoid the area based on the displayed boundary zone 740. The boundary zone 740 may be displayed as a mesh, as a holographic object, as a transparent object, as a semitransparent object, and/or as a solid object. The boundary zone 740 may be displayed as a color, a gradient of colors (changing color as an object approaches the boundary zone 740), and/or as a shading.

The robotic surgical system 10 may prevent robotic arms 40 from entering the boundary zone via movement limits set on the motors that control the robotic arms 40. In particular, the kinematic pipeline providing movement commands based on user inputs is limited by the boundary zone 740, i.e., the robotic arms 40 may be stopped and/or slowed when a portion of robotic arms 40 is moved outside the boundary zone 740. In particular, the controller 602 may cause the robotic surgical system 10 to receive or access 3D volumetric data of a robotic arm of a robotic surgical system 10 in the real-world environment and receive or access a spatial position of the robotic arm 40. This may be accomplished by sending the 3D volumetric data to the control tower 20 via data distribution service (DDS) message or another publish-subscribe communication protocol. The main controller 21a may then, based on the 3D volumetric data, prevent the robot arm 40 from entering the boundary zones 740, and apply force feedback to the input devices at the surgical console 30 resisting control motion in that direction. For example, the controller 21a may cause the robotic surgical system 10 to determine that the robotic arm is crossing a threshold of the first boundary zone 740 based on the spatial position and the 3D volumetric data and stop the robotic arm from entering the first boundary zone 740.

In addition to controlling the movement of the robotic arms 40, the controller 21a may output alerts in response to the portion of robotic arms 40 is moved outside the boundary zone 740. The alert may be audio or visual and may be displayed on the augmented reality device 600 and/or the displays 23, 32, and/or 34.

FIG. 7 is an image of the composite view 700 of the clinical workspace augmentation displaying a boundary zone 740. Users 704 are shown generating a 3D boundary zone 740 using the methods described herein. One or more mobile carts 60a-b may be positioned around a surgical table 90. The robotic surgical system 10 may prevent the robotic arms 40 of the mobile carts 60a-b from entering the boundary zone(s) 740.

It will be understood that various modifications may be made to the aspects disclosed herein. Therefore, the above description should not be construed as limiting but merely as exemplifications of various aspects. Those skilled in the art will envision other modifications within the scope and spirit of the claims appended thereto.

What is claimed is:

1. A computer-implemented method for clinical workspace augmentation, the method comprising:
capturing a real-world environment by an imaging device, wherein the real-world environment includes an input object;
identifying the input object in the captured real-world environment;
tracking a path of the input object in the captured real-world environment;
generating at least one boundary zone based on the tracked path of the input object in the captured real-world environment;
rendering an overlay including the at least one boundary zone; and
displaying a composite view of the captured real-world environment and the overlay on a first display of an augmented reality device.

2. The computer-implemented method of claim 1, wherein the input object includes at least one of a hand of a user or an input controller.

3. The computer-implemented method of claim 1, wherein tracking the path of the input object is based on at least one of a gesture, pose recognition, eye gaze tracking, and/or voice command.

4. The computer-implemented method of claim 1, wherein identifying the input object in the captured real-world environment is based on object detection.

5. The computer-implemented method of claim 4, wherein the object detection is performed by:
generating a spatial mesh based on the captured real-world environment;
determining boundaries of the input object based on the spatial mesh; and
identifying the input object based on a machine learning model, where the determined boundaries are provided as an input to the machine learning model.

6. The computer-implemented method of claim 1, further comprising modifying the at least one boundary zone by:
selecting a generated shape configured to become a portion of the at least one boundary zone;
positioning the generated shape in the captured real-world environment;
receiving input to transform the generated shape by at least one of moving, rotating, or scaling the generated shape; and
adding the transformed generated shape to the at least one boundary zone.

7. The computer-implemented method of claim 1, further comprising modifying the at least one boundary zone by:
selecting a plurality of waypoints to become a portion of the at least one boundary zone;
receiving input to position the plurality of waypoints;
connecting the waypoints from a second boundary zone; and
adding the second boundary zone to the at least one boundary zone.

8. The computer-implemented method of claim 1, further comprising:
receiving 3D volumetric data of a robotic arm of a robotic surgical system in the real-world environment;
receiving a spatial position of the robotic arm;
determining that the robotic arm is crossing a threshold of the at least one boundary zone based on the spatial position and the 3D volumetric data; and
stopping the robotic arm from entering the at least one boundary zone.

9. The computer-implemented method of claim 8, further comprising:
providing force feedback to an input device of a surgical console of the robotic surgical system in response to the determining that the robotic arm is crossing the threshold of the at least one boundary zone.

10. The computer-implemented method of claim 1, further comprising:
displaying on a second display a composite view of the at least one boundary zone and the real-world environment from a point of view of the second display, wherein the point of view of the second display is different than a point of view of the first display.

11. A system for clinical workspace augmentation, the system comprising:
an augmented reality device including:
an imaging device configured to capture images of a real-world environment;
a display configured to display a composite view;
a processor; and
a memory, including instructions stored thereon which, when executed by the processor, cause the system to:
identify an input object in the captured real-world environment;
track a path of the input object in the captured real-world environment;
generate at least one boundary zone based on the tracked path of the input object in the captured real-world environment;
render an overlay including the at least one boundary zone; and
display a composite view of the captured real-world environment and the overlay on the display of the augmented reality device.

12. The system of claim 11, wherein the input object includes at least one of a hand of a user or an input controller.

13. The system of claim 11, wherein tracking the path of the input object is based on at least one of a gesture or pose recognition.

14. The system of claim 11, wherein identifying the input object in the captured real-world environment is based on object detection.

15. The system of claim 14, wherein the object detection is performed by:
generating a spatial mesh based on the captured real-world environment;
determining boundaries of the input object based on the spatial mesh; and
identifying the input object based on a machine learning model, where the determined boundaries are provided as an input to the machine learning model.

16. The system of claim 11, wherein the instructions, when executed by the processor, further cause the system to modify the at least one boundary zone by:
selecting a generated shape configured to become a portion of the at least one boundary zone;
positioning the generated shape in the captured real-world environment;
receiving input to transform the generated shape by at least one of moving, rotating, or scaling the generated shape; and
adding the transformed generated shape to the at least one boundary zone.

17. The system of claim 11, wherein the instructions, when executed by the processor, further cause the system to modify the at least one boundary zone by:
selecting a plurality of waypoints to become a portion of the at least one boundary zone;
receiving input to position the plurality of waypoints;
connecting the waypoints from a second boundary zone; and
adding the second boundary zone to the at least one boundary zone.

18. The system of claim 11, wherein the instructions, when executed by the processor, further cause the system to:
receive 3D volumetric data of a robotic arm of a robotic surgical system in the real-world environment;
receive a spatial position of the robotic arm;
determine that the robotic arm is crossing a threshold of the at least one boundary zone based on the spatial position and the 3D volumetric data; and
stop the robotic arm from entering the at least one boundary zone.

19. The system of claim 18, wherein the instructions, when executed by the processor, further cause the system to:
provide force feedback to an input device of a surgical console of the robotic surgical system in response to the determining that the robotic arm is crossing the threshold of the at least one boundary zone.

20. A non-transitory computer-readable medium storing instructions which, when executed by a processor, cause the processor to perform a method comprising:
capturing a real-world environment by an imaging device, wherein the real-world environment includes an input object;
identifying the input object in the captured real-world environment;
tracking a path of the input object in the captured real-world environment;
generating at least one boundary zone based on the tracked path of the input object in the captured real-world environment;
rendering an overlay including the at least one boundary zone; and
displaying a composite view of the captured real-world environment and the overlay on a display of an augmented reality device.
